(19) United States
(12) Patent Application Publication
(10) Pub. No.: US 2024/0058334 A1
(43) Pub. Date: Feb. 22, 2024

(54) METHODS FOR TREATING SMALL CELL LUNG CANCER AND OTHER NEUROENDOCRINE CANCERS

(71) Applicant: AstraZeneca AB, Södertälje (SE)

(72) Inventors: Lauren A. BYERS, Houston, TX (US); Carl M. GAY, Houston, TX (US)

(21) Appl. No.: 18/549,852

(22) PCT Filed: Mar. 10, 2022
(86) PCT No.: PCT/US2022/019763

§ 371 Date: Sep. 8, 2023

Related U.S. Application Data
(60) Provisional application No. 63/159,401, filed on Mar. 10, 2021.

(51) Int. Cl. A61K 31/4985; G01N 33/574; A61K 31/5377; A61K 31/497; A61P 35/00

(52) U.S. Cl. CPC A61K 31/4985; G01N 33/57423; A61K 31/5377; A61K 31/497; A61P 35/00

(57) ABSTRACT

The current disclosure provides novel therapeutic methods for treating SCLC and other neuroendocrine cancers by evaluating the biomarker SLFN11. Aspects of the disclosure relate to a method for treating a subject with small cell lung cancer (SCLC) or with a neuroendocrine cancer, the method comprising administering a combination of lurbinectedin and an ATR inhibitor to a subject that has had been determined to be negative for SLFN11 expression or have low SLFN11 expression in a biological sample from the subject.

### I. FIELD OF THE INVENTION

The present invention relates generally to the fields of molecular biology and therapeutic diagnosis. More particularly, it concerns methods and compositions involving prognosing, diagnosing, and treating SCLC and other neuroendocrine cancers.

### II. BACKGROUND

Small cell lung cancer (SCLC) is an aggressive subtype of lung cancer, accounting for approximately 15% of all lung cancer cases in United States. SCLC is characterized by small cells with poorly defined cell borders and minimal cytoplasm, rare nucleoli, and finely granular chromatin. Due to the aggressive nature of the disease, the low rate of early diagnosis, and the lack of effective therapies, prognosis is generally poor. Median survival time from diagnosis for untreated SCLC patients is only two to four months. When chemotherapy and/or radiation modalities are used, the initial response rate to among SCLC patients is high (approximately 60 to 80%), but relapse occurs in the majority treated patients, who then are largely refractory to further systemic therapy. Thus, even with current treatment modalities, the median survival time for patients with limited-stage disease is 16 to 24 months and for patients with extensive disease, seven to 12 months. To improve patient survival rates, it is essential to treat patients with chemotherapeutic agents to which their tumors are sensitive. Use of targeted drugs in the treatment of SCLC represents a major unmet medical need. Unlike non-small cell lung cancers (NSCLC), there are currently no targeted therapies with demonstrated benefit for patients with this disease. Thus, there is a need to align SCLC patients with suitable treatments based on their individual genetic profiles. Understanding a given tumor's genetic profile will also enable early diagnosis, detection, and treatment selection.

### SUMMARY

The current disclosure provides novel therapeutic methods for treating SCLC negative or SCLC low expressing neuroendocrine cancers. Aspects of the disclosure relate to a method for treating a subject with small cell lung cancer (SCLC) or with a neuroendocrine cancer, the method comprising administering a combination of lurbinectedin and an ATR (taxia telangiectasia and Rad3-related protein) inhibitor to a subject that has had been determined to be negative for SLFN11 expression or have low SLFN11 expression in a biological sample from the subject. Further aspects relate to a method for predicting a response to lurbinectedin and an ATR inhibitor in a subject having a neuroendocrine cancer or SCLC comprising a) evaluating SLFN11 in a biological sample from the subject; b) predicting that the subject will respond to the combination of lurbinectedin and ATR inhibitor after (i) SLFN11 expression is not detected in the biological sample from the patient; (ii) the patient is determined to have low or substantially the same level of SLFN11 expression compared to a control wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04; or (iii) iii) the H-score for the level of expression in the biological sample from the subject is less than 1.

In some aspects, the ATR inhibitor comprises or consists of ceralasertib (AZD6738) and/or berzosertib (VX-970). In some aspects, the ATR inhibitor comprises or consists of ceralasertib. In some aspects, the ATR inhibitor comprises or consists of berzosertib. IN some aspects, the ATR inhibitor is selected from VE-821, LR-02, RP-3500, SC-0245, M-1774, M4344, ATG-018, IMP-9064, nLs-BG-129, BAY-1895344, berzosertib, ART-0380, ATRN-119, ATRN-212, BKT-300, AZ-20, ceralasertib, and combinations thereof. The cancer may be a SCLC or neuroendocrine cancer or the subject may be one that has been diagnosed or determined to have SCLC or a neuroendocrine cancer. In some aspects, the subject was evaluated for, or the methods comprise evaluating or determining SLFN11 expression by evaluating SLFN11 in an immunohistochemistry assay performed on a biological sample from the subject. In some aspects, the biological sample comprises a blood, serum, plasma, biopsy, or tissue sample. The biological sample may also be a biological sample described herein. In some aspects, the biological sample comprises circulating tumor cells. In some aspects, the biological sample comprises circulating tumor DNA. In some aspects, the expression level of SLFN11 in the biological sample from the subject has been quantitated or the method further comprises quantitating the expression level of SLFN11. In some aspects, the expression level of SLFN11 is normalized or the method further comprises normalizing the expression level of SLFN11. In some aspects, the subject has been determined to be negative for SLFN11 expression in the biological sample. In some aspects, the subject has been determined to have SLFN11-negative cells in the biological sample from the subject. In some aspects, the subject has or has been determined to have a low level of SLFN11 expression in the biological sample from the subject. In some aspects, the subject has or has been determined to have a low or substantially the same level of SLFN11 expression in the biological sample from the subject compared to a control level of expression. In some aspects, the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04. In some aspects, the control may be the expression level of SLFN11 in non-cancerous cells, aspect In some aspects, the control comprises a cut-off value above which, high expression of SLFN11 is defined and below which, low expression of SLFN11 is defined. In some aspects, the cut-off value is further defined as an H-score, which is defined as the percentage of cells expressing SLFN11 times the intensity of SLFN11 staining (0 [meaning no staining/expression] vs 1+/2+/3+ for a range of possible H-scores of 0-300). In some aspects, the H-score is 1. In some aspects, the H-score is at least, H-score is at most, or H-score is exactly 0.1, 0.5, 0.8, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any derivable range therein. Further aspects are described in Allison Stewart C, et al., Oncotarget. 2017 Apr. 25; 8(17):28575-28587 and Pietanza M C, et al., LA. J Clin Oncol. 2018 Aug. 10; 36(23):2386-2394, which are herein incorporated by reference. In some aspects, the methods further comprise determining the H-score for SLFN11 expression in the subject. In some aspects, the subject is determined to have a H-score that is less than 1. In some aspects, the subject is determined to have a H-score of 1 or less.

In some aspects, the subject has not previously been treated with lurbinectedin. In some aspects, the cancer is further defined as recurrent. In some aspects, the cancer comprises SCLC type A. The subject may be a human, mammalian, non-human primate, rat, mouse, pig, horse, cat, or dog. In some aspects, the subject is a human.

In some aspects, the expression level of the measured biomarkers are/were determined to be significantly different as compared to a control level of expression, wherein the control level of expression comprises the level of expression of the biomarkers in a cancer that is not a SCLC or neuroendocrine cancer. In some aspects, the expression level of the measured biomarkers are/were determined to be not significantly different as compared to a control level of expression, wherein the control level of expression comprises the level of expression of the biomarkers in a non-cancerous sample. In some aspects, the expression level of the measured biomarkers are/were determined to be significantly different as compared to a control level of expression, wherein the control level of expression comprises the level of expression of the biomarkers in a non-cancerous sample. In some aspects, the expression level of the measured biomarkers are/were determined to be not significantly different as compared to a control level of expression, wherein the control level of expression comprises the level of expression of the biomarkers in a cancerous sample, wherein the cancer comprises a cancer described herein. In some aspects, the expression level of the measured biomarkers are/were determined to be significantly different as compared to a control level of expression, wherein the control level of expression comprises the level of expression of the biomarkers in a cancerous sample, wherein the cancer comprises a cancer described herein.

In some aspects, the one or more biomarkers has an absolute value of the signature weight of greater than 0.025. The term “absolute value” refers to the magnitude of a real number without regard to its sign. Signature weights are listed in Tables 1-3. In some aspects, the one or more biomarkers has an absolute value of the signature weight of greater than or less than 0.000001, 0.000002, 0.000003, 0.000004, 0.000005, 0.000006, 0.000007, 0.000008, 0.000009, 0.00001, 0.000015, 0.00002, 0.000025, 0.00003, 0.000035, 0.00004, 0.000045, 0.00005, 0.000055, 0.00006, 0.000065, 0.00007, 0.000075, 0.00008, 0.000085, 0.00009, 0.000095, 0.0001, 0.00015, 0.0002, 0.00025, 0.0003, 0.00035, 0.0004, 0.00045, 0.0005, 0.00055, 0.0006, 0.00065, 0.0007, 0.00075, 0.0008, 0.00085, 0.0009, 0.00095, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, or 0.05 (or any range derivable therein).

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect or embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments and aspects discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows that cells with low expression of SLFN11 have higher lurbinectedin IC₅₀ values.

FIG. 2 shows results from treatment with lurbinectedin combined with AZD673 in SCLC cell lines H211, H446, and SHP77, demonstrating synergism between lurbinectedin and AZD673.

FIG. 3A-3C show results demonstrating that the combination effect of lurbinectedin and AZD6738 also reflects in intracellular DNA damage markers.

FIG. 4A-4C. Combination of lurbinectedin with ATR inhibitor is greater than additive in SLFN11 low cell lines. A, Relative proliferation of cell lines representing greater than additive, and additive responses following 96 h treatment with lurbinectedin (Lurbi), ceralasertib and their combination at indicated concentrations (mean±SEM). B. Bar-graph representing ΔAUC for all 21 cell lines tested, color coded by SCLC subtype. ΔAUC value denotes difference in area under the dose-response curve of the observed drug combination and the predicted additive effect of the single agents was calculated using the BLISS independence model. ΔAUC<−0.1 indicates a greater than additive effect of the combination. C. Western-blot showing changes in pCHK1, pγH2Ax and cleaved caspase3 in H211 and H865 cell lines treated with 0.6 nM of lurbinectedin, 0.3 μM of ceralasertib and their combination.

FIG. 5A-5D. A. Relative proliferation of cell lines representing greater than additive, and additive responses following 96 h treatment with lurbinectedin (Lurbi), VX-970 and their combination at indicated concentrations (mean±SEM). B. Bar-graph representing ΔAUC for all SCLC cell lines tested, color coded by SCLC subtype. C. Western-blot showing changes in pCHK1, pγH2Ax and cleaved caspase3 in H211 and H865 cell lines treated with 0.6 nM of lurbinectedin, 0.3 μM of VX-970 and their combination. D. Comparison of delta AUC values for the combination of lurbinectedin and cerelasertib between cell lines from the four SCLC subtypes SCLC-A, -N, -P and -I (p-values by student's t-test).

### DETAILED DESCRIPTION OF THE INVENTION

### I. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from lung tissue by any of the biopsy methods previously mentioned. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects, the sample comprises a fractionated sample, such as a blood sample that has been fractionated by centrifugation or other fractionation technique. The sample may be enriched in white blood cells or red blood cells. In some aspects, the sample may be fractionated or enriched for leukocytes or lymphocytes. In some aspects, the sample comprises a whole blood sample.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a esophageal or a suspected esophageal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### II. Cancer Monitoring

In certain aspects, the methods of the disclosure may be combined with one or more other cancer diagnosis or screening tests at increased frequency if the patient is determined to be at high risk for recurrence or have a poor prognosis based on the biomarker expression described above.

In some aspects, the methods of the disclosure further include one or more monitoring tests. The monitoring protocol may include any methods known in the art. In particular, the monitoring include obtaining a sample and testing the sample for diagnosis. For example, the monitoring may include endoscopy, biopsy, endoscopic ultrasound, X-ray, barium swallow, a Ct scan, a MRI, a PET scan, laparoscopy, or cancer biomarker testing. In some aspects, the monitoring test comprises radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

Methods of the disclosure may further include one or more of a urinalysis, urine cytology, urine culture, or urine tumor marker tests. Different urine tests look for specific substances made by cancer cells. One or more of these tests may be used in the methods of the disclosure. These include the tests called NMP22® (or BladderChek®), BTA Stat®, Immunocyt®, and UroVysion®. Methods of the disclosure also include cystoscopy. In this method, a urologist uses a cystoscope, which is a long, thin, flexible tube with a light and a lens or a small video camera on the end. Fluorescence cystoscopy (also known as blue light cystoscopy) may be done along with routine cystoscopy. For this exam, a light-activated drug is put into the bladder during cystoscopy. It's taken up by cancer cells. When the doctor then shines a blue light through the cystoscope, any cells containing the drug will glow (fluoresce). This can help the doctor see abnormal areas that might have been missed by the white light normally used.

Methods of the disclosure also include the use of transurethral resection of bladder tumor (TURBT). The procedure used to biopsy an abnormal area is a transurethral resection of bladder tumor (TURBT), also known as just a transurethral resection (TUR). During this procedure, the doctor removes the tumor and some of the bladder muscle around the tumor. The removed samples are then sent to a lab to look for cancer. Bladder cancer can sometimes start in more than one area of the bladder (or in other parts of the urinary tract). Because of this, the doctor may take samples from many different parts of the bladder, especially if cancer is strongly suspected but no tumor can be seen. Salt water washings of the inside the bladder may also be collected and tested for cancer cells.

In some aspects, imaging tests are performed or the subject is one that has undergone an imaging test. Imaging tests may use x-rays, magnetic fields, sound waves, or radioactive substances. In some aspects, the imaging test comprises an Intravenous pyelogram (IVP). An intravenous pyelogram (IVP), also called an intravenous urogram (IVU), is an x-ray of all of the urinary system taken after injecting a special dye into a vein. This dye is removed from the bloodstream by the kidneys and then passes into the ureters and bladder. X-rays are done while this is happening. The dye outlines these organs on the x-rays and helps show urinary tract tumors. In some aspects, the imaging test comprises a retrograde pyelogram. For this test, a catheter (thin tube) is put in through the urethra and up into the bladder or into a ureter. Then a dye is injected through the catheter to make the lining of the bladder, ureters, and kidneys easier to see on x-rays. In some aspects, the imaging test comprises computed tomography (CT) scan. A CT scan uses x-rays to make detailed cross-sectional pictures of the body. CT-guided needle biopsy: CT scans can also be used to guide a biopsy needle into a suspected tumor. This can be used to take samples from areas where the cancer may have spread. In some aspects, the imaging test comprises magnetic resonance imaging (MRI) scan. Like CT scans, MRI scans show detailed images of soft tissues in the body. But MRI scans use radio waves and strong magnets instead of x-rays. In some aspects, the imaging test comprises an ultrasound. Ultrasound uses sound waves to create pictures of internal organs. Ultrasound can also be used to guide a biopsy needle into a suspected area of cancer in the abdomen or pelvis. In some aspects, the imaging test comprises a chest x-ray or bone scan. A chest x-ray or bone scan may be done to see if the bladder cancer has spread to the lungs or bone, respectively.

### III. ROC analysis

In statistics, a receiver operating characteristic (ROC), or ROC curve, is a graphical plot that illustrates the performance of a binary classifier system as its discrimination threshold is varied. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1−specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig MH, Campbell G (1993) Receiver-operating characteristic (ROC) plots: a fundamental evaluation tool in clinical medicine. Clinical Chemistry 39:561-577; and Greiner M, Pfeiffer D, Smith RD (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### IV. Nucleic Acid Assays

Aspects of the methods include assaying nucleic acids to determine expression or activity levels and/or the presence of CXCL13 expressing cells and/or ARID1A mutant cells in a biological sample. Arrays can be used to detect differences between two samples. Specifically contemplated applications include identifying and/or quantifying differences between RNA from a sample that is normal and from a sample that is not normal, between a cancerous condition and a non-cancerous condition. Also, RNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

To determine expression levels of a biomarker, an array may be used. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

Further assays useful for determining biomarker expression include, but are not limited to, nucleic amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

A further assay useful for quantifying and/or identifying nucleic acids, such as nucleic acids comprising biomarker genes, is RNAseq. RNA-seq (RNA sequencing), also called whole transcriptome shotgun sequencing, uses next-generation sequencing (NGS) to reveal the presence and quantity of RNA in a biological sample at a given moment in time. RNA-Seq is used to analyze the continually changing cellular transcriptome. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

### V. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, flow cytometry, or immunofluorescence techniques to detect biomarker expression such as CXCL13. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 1251, 3H, and 1311. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

### VI. Administration of Therapeutic Compositions

The therapy provided herein may comprise administration of a combination of therapeutic agents, such as a first anticancer therapy and a second anticancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. Methods of Treatment

Provided herein are methods for treating or delaying progression of cancer in an subject through the administration of therapeutic compositions.

In some aspects, the therapies result in a sustained response in the individual after cessation of the treatment. The methods described herein may find use in treating conditions where enhanced immunogenicity is desired such as increasing tumor immunogenicity for the treatment of cancer.

In some aspects, the individual has cancer that is resistant (has been demonstrated to be resistant) to one or more anticancer therapies. In some aspects, resistance to anticancer therapy includes recurrence of cancer or refractory cancer. Recurrence may refer to the reappearance of cancer, in the original site or a new site, after treatment. In some aspects, resistance to anticancer therapy includes progression of the cancer during treatment with the anticancer therapy. In some aspects, the cancer is at early stage or at late stage.

In some aspects of the methods of the present disclosure, the cancer has low levels of T cell infiltration. In some aspects, the cancer has no detectable T cell infiltrate. In some aspects, the cancer is a non-immunogenic cancer (e.g., non-immunogenic colorectal cancer and/or ovarian cancer). Without being bound by theory, the combination treatment may increase T cell (e.g., CD4+ T cell, CD8+ T cell, memory T cell) priming, activation, proliferation, and/or infiltration relative to prior to the administration of the combination.

The cancer may be a solid tumor, metastatic cancer, or non-metastatic cancer. In certain aspects, the cancer may originate in neuroendocrine cells.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

In certain aspects, further cancer or metastasis examination or screening, or further diagnosis such as contrast enhanced computed tomography (CT), positron emission tomography-CT (PET-CT), and magnetic resonance imaging (MRI) may be performed for the detection of cancer or cancer metastasis in patients determined to have a certain gut microbiome composition.

Methods of the disclosure relate to the treatment of subjects with cancer. In some aspects, the methods may be employed with respect to individuals who have tested positive for such cancer, who have one or more symptoms of a cancer, or who are deemed to be at risk for developing such a cancer.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### IX. Example 1—Treatment of SLFN11-Negative or Low Small Cell Lung Cancer with the Combination of Lurbinectedin and ATR Inhibitors

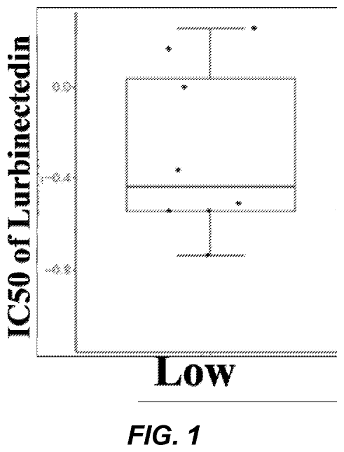

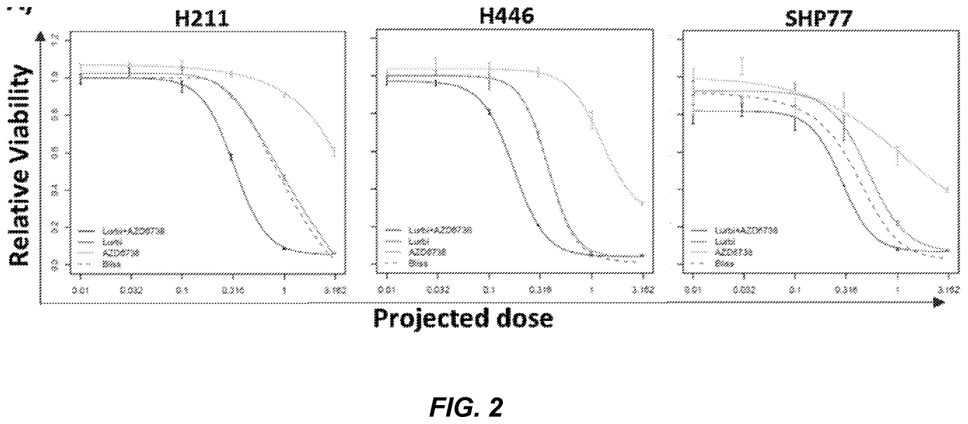

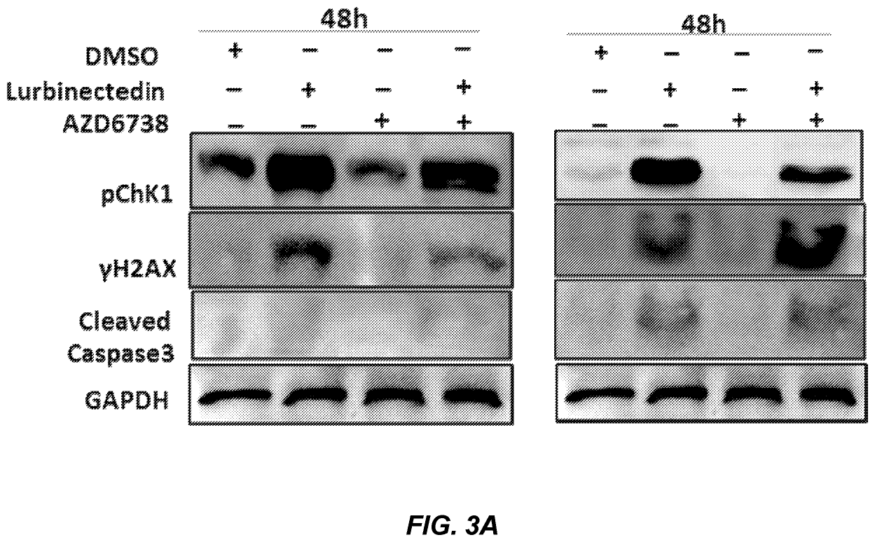

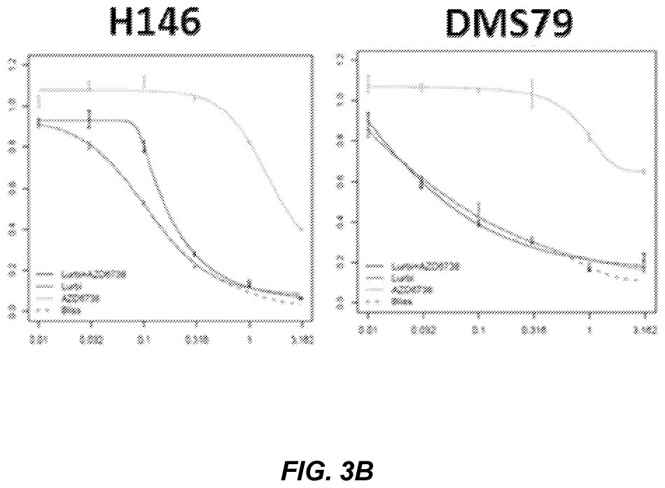

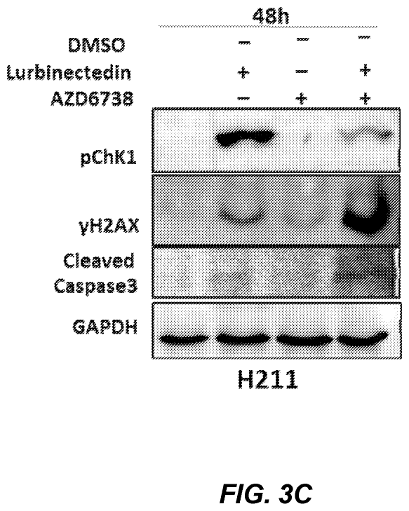

Effect of lurbinectedin in combination with ATR inhibitor AZD6738 was checked in 96 hr proliferation assays on those cell lines. Treatment with lurbinectedin combined with ATR kinase inhibitor AZD6738 showed synergistic effect in SCLC cell lines with higher IC₅₀ values for lurbinectedin (FIGS. 1-3).

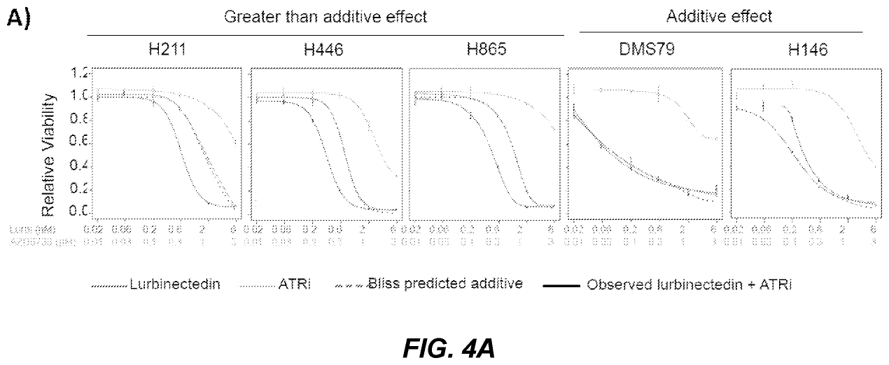

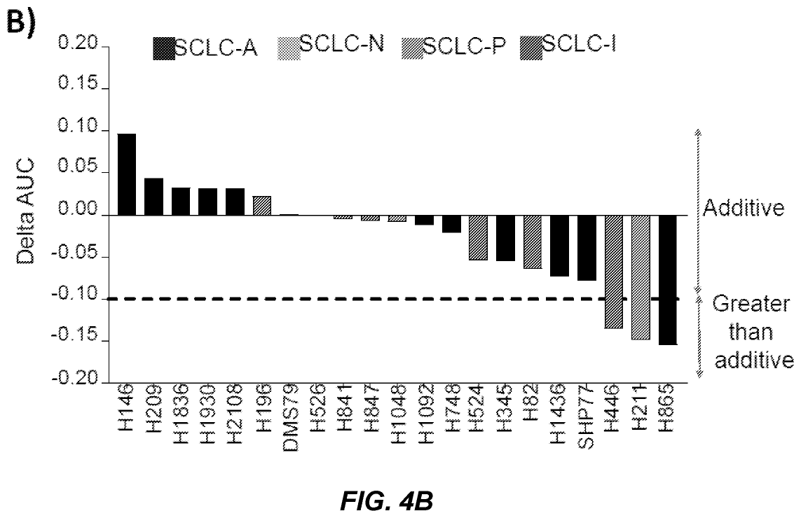

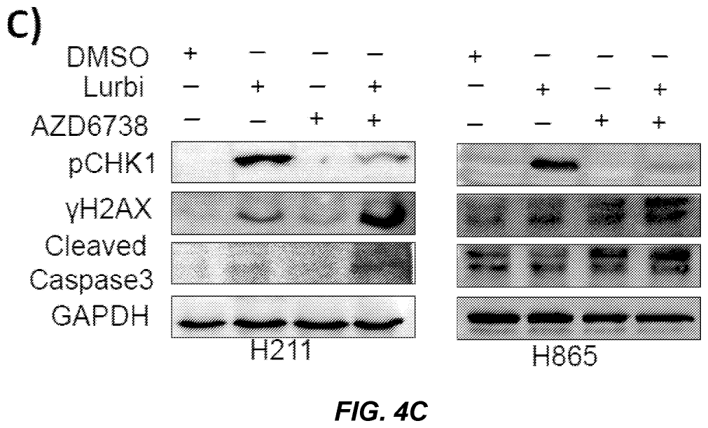

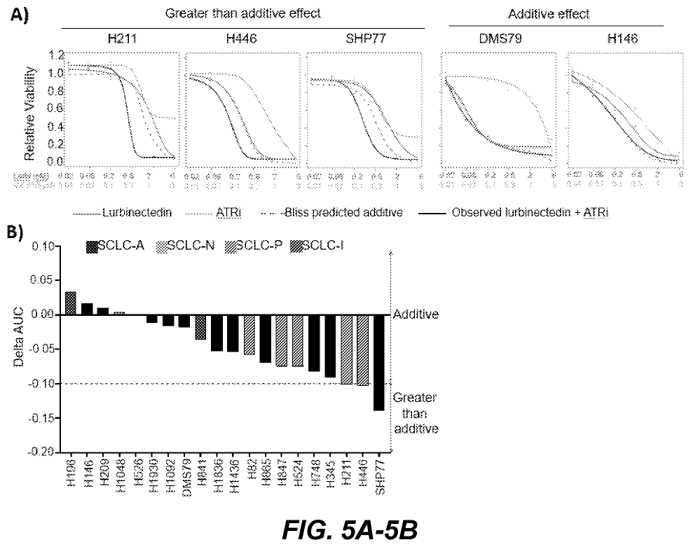

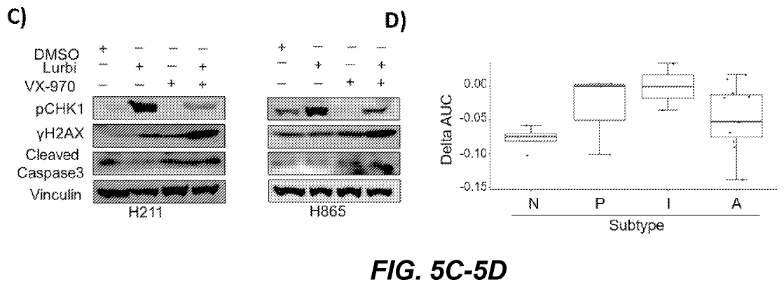

The inventors further investigated the combination of ceralasertib (AZD6738) and berzosertib, with lurbinectedin in SCLC cell lines. Using the Bliss Delta AUC (ΔAUC) method, which compares the observed and predicted additive interaction between two drugs, the inventors observed a range of interactions (FIG. 4A, 5A), both additive (ΔAUC>-0.1 and <0.1) and greater than additive (Δ<−0.1). The three cell lines with a greater than additive response to the combination with ceralasertib—H865, H211, and H446—are members of subsets A, P, and N respectively (FIG. 4B) and as expected, are amongst the cell lines with low SLFN11 level and least sensitive to single agent lurbinectedin. To account for potential off-target effects, we also tested lurbinectedin with a second ATR inhibitor, berzosertib (VX-970). Similar to the combination with ceralasertib, the lurbinectedin/berzosertibcombination showed a range of interactions, with the three cell lines with a greater than additive response −SHP77, H211 and H446 represent the A, P, and N subtypes respectively (FIG. 5B). The inventors found that cell lines with lower SLFN11 expression had better responses to the combination of lurbinectedin and ceralasertib or berzosertib. All three cell lines in which a greater than additive response to the combination of lurbinectedin and ceralasertib or berzosertib was observed were in the SLFN11 low group, and were relatively resistant to single agent ceralasertib or berzosertib. To understand the mechanism of action when we treated H211 and H865 cell lines with the combination of lurbinectedin and ceralasertib or berzosertib for 48 h, Western blotting results showed marked increase in γH2AX and cleaved caspase3 levels following the combination treatment as compared to either single agent lurbinectedin and ceralasertib (FIG. 4C) and for berzosertib (FIG. 5C). Reduction in pCHK1 in single agent ATR inhibitor and in combination treatment group in compared to lurbinectedin represent halt in DNA damage repair system (FIG. 5C). There appear to be no differences in effectiveness of combining lurbinectedin with ceralasertib or berzosertib between the SCLC-A, -N, -P, and -I subtypes (FIG. 5D). This data shows that SLFN11 low SCLC cells are sensitive to treatment with the combination of lurbinectedin and ceralasertib or berzosertib.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for treating a subject with small cell lung cancer (SCLC) or with a neuroendocrine cancer, the method comprising administering a combination of lurbinectedin and an ATR (taxia telangiectasia and Rad3-related protein) inhibitor to a subject that has had been determined to be negative for SLFN11 expression or have low SLFN11 expression in a biological sample from the subject.

2. The method of claim 1, wherein the ATR inhibitor comprises VE-821, LR-02, RP-3500, SC-0245, M-1774, M4344, ATG-018, IMP-9064, nLs-BG-129, BAY-1895344, berzosertib, ART-0380, ATRN-119, ATRN-212, BKT-300, AZ-20, ceralasertib, or combinations thereof.

3. The method of claim 2, wherein the ATR inhibitor comprises ceralasertib (AZD6738) or berzosertib (VX-970).

4. The method of any one of claims 1-3, wherein the subject was evaluated for SLFN11 expression by evaluating SLFN11 in an immunohistochemistry assay performed on a biological sample from the subject.

5. The method of claim 1 or 4, wherein the biological sample comprises a blood, serum, plasma, liquid biopsy, pleural effusion, biopsy, or tissue sample.

6. The method of claim 5, wherein the biological sample comprises circulating tumor cells.

7. The method of any one of claims 1-6, wherein the biological sample comprises circulating tumor DNA.

8. The method of any one of claims 1-7, wherein the expression level of SLFN11 in the biological sample from the subject has been quantitated.

9. The method of claim 8, wherein the expression level of SLFN11 is normalized.

10. The method of claim 1, wherein the subject has been determined to have SLFN11-negative cells in the biological sample from the subject.

11. The method of any one of claims 1-10, wherein the subject has or has been determined to have a low level of SLFN11 expression in the biological sample from the subject.

12. The method of any one of claims 1-11, wherein the subject has or has been determined to have a low or substantially the same level of SLFN11 expression in the biological sample from the subject compared to a control level of expression.

13. The method of claim 12, wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04.

14. The method of any one of claims 10-13, wherein the subject has been determined to have a H-score for SLFN11 of less than 1.

15. The method of claim 14, wherein the subject has been determined to have a H-score for SLFN11 of less than 0.5.

16. The method of any one of claims 1-15, wherein the subject has not previously been treated with lurbinectedin.

17. The method of any one of claims 1-16, wherein the cancer is further defined as recurrent.

18. The method of any one of claims 1-17, wherein the cancer comprises SCLC type A.

19. The method of any one of claim 1-18, wherein the subject is a human subject.

20. A method for predicting a response to lurbinectedin and an ATR inhibitor in a subject having a neuroendocrine cancer or SCLC comprising
a) evaluating SLFN11 in a biological sample from the subject;
b) predicting that the subject will respond to the combination of lurbinectedin and ATR inhibitor after (i) SLFN11 expression is not detected in the biological sample from the patient; (ii) the patient is determined to have low or substantially the same level of SLFN11 expression compared to a control wherein the control represents the level of expression of SLFN11 in lurbinectedin insensitive cells or the level of expression of SLFN11 in cells in which the pIC50 of lurbinectedin is greater than −0.08, −0.07, −0.06, −0.05, or −0.04; or (iii) iii) the H-score for the level of expression in the biological sample from the subject is less than 1.

21. The method of claim 20, wherein the subject was evaluated for SLFN11 expression by evaluating SLFN11 in an immunohistochemistry assay performed on a biological sample from the subject.

22. The method of claim 20 or 21, wherein the biological sample comprises a blood, serum, plasma, liquid biopsy, pleural effusion, biopsy, or tissue sample.

23. The method of claim 22, wherein the biological sample comprises circulating tumor cells.

24. The method of any one of claims 20-23, wherein the biological sample comprises circulating tumor DNA.

25. The method of any one of claims 20-24, wherein the expression level of SLFN11 in the biological sample from the subject has been quantitated.

26. The method of claim 25, wherein the expression level of SLFN11 is normalized.

27. The method of any one of claims 20-26, wherein the method further comprises treating the subject.

28. The method of claim 27, wherein the subject is treated with the combination of lurbinectedin and an ATR inhibitor.

29. The method of any one of claims 20-28, wherein the ATR inhibitor comprises VE-821, LR-02, RP-3500, SC-0245, M-1774, M4344, ATG-018, IMP-9064, nLs-BG-129, BAY-1895344, berzosertib, ART-0380, ATRN-119, ATRN-212, BKT-300, AZ-20, ceralasertib, or combinations thereof.

30. The method of claim 29, wherein the ATR inhibitor comprises ceralasertib (AZD6738) or berzosertib (VX-970).

31. The method of any one of claims 20-30, wherein the subject has not previously been treated with lurbinectedin.

32. The method of any one of claims 20-31, wherein the cancer is further defined as recurrent.

33. The method of any one of claims 20-32, wherein the cancer comprises SCLC type A.

34. The method of any one of claim 20-33, wherein the subject is a human subject.
